(19) United States
(12) Patent Application Publication
(10) Pub. No.: US 2005/0129620 A1
(43) Pub. Date: Jun. 16, 2005

(54) Metering valve and pharmaceutical metered dose inhaler and methods thereof

(76) Inventor: Verna Lo Clark, Durham, NC (US)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/497,947

(22) PCT Filed: Dec. 6, 2002
(86) PCT No.: PCT/US02/38874

Related U.S. Application Data
(60) Provisional application No. 60/338,190, filed on Dec. 7, 2001.

(51) Int. Cl. A61L 9/04; A61M 11/00

(52) U.S. Cl. 424/45; 128/200.23; 427/2.14

(57) ABSTRACT

Methods of treating an elastomeric substrate that include, in any suitable order, providing an elastomeric substrate in a bath including an alcohol and an alkaline material at a bath temperature effective for treatment, providing ultrasonic energy at a treatment-effective frequency and power level to the bath for a time period sufficient to treat the elastomeric substrate, rinsing the treated elastomeric substrate with de-ionized water, and drying the rinsed and treated elastomeric substrate are described.

### FIELD OF THE INVENTION

The invention generally applies to methods and articles of manufacture made therefrom of treating and coating elastomeric substrate materials, particularly elastomers fabricated into seals and gaskets for use in pharmaceutical inhalation devices, particularly metered dose inhalers.

The invention provides a container for a metered dose inhaler (MDI) for use in dispensing a quantity of a medicament-containing formulation which may be used in the treatment of respiratory disorders.

A preferred medicament is 4-hydroxy-α′-[[[6-(4-phenylbutoxy)hexyl]amino]methyl]-1,3-benzenedimethanol which was described as one of a wide range of bronchodilators in GB-A-2140800. This compound is also known by the generic name of salmeterol, the 1-hydroxy-2-naphthoate (xinafoate) salt of which has become widely known as a highly effective treatment of inflammatory diseases, such as asthma and chronic obstructive pulmonary disease (COPD).

Containers for aerosol formulations commonly comprise a vial body (can or canister) coupled to a valve. The valve comprises a valve stem through which the formulations are dispensed. Generally the valve includes a rubber valve seal intended to allow reciprocal movement of the valve stem which prevents leakage of propellant from the container. Metered dose inhalers comprise a valve which is designed to deliver a metered amount of an aerosol formulation, to the recipient, per actuation. Such a metering valve generally comprises a metering chamber which is of a set volume which aims to administer per actuation an accurate, predetermined dose.

Metering valves incorporate gaskets (also referred to as seals) to prevent leakage of propellant from the valve. The gasket may comprise suitable elastomeric material such as for example low density polyethylene, chlorobutyl, black and white butadiene-acrylonitrile rubbers, butyl rubber and neoprene.

Valves for use in MDIs are available from manufacturers well known in the aerosol industry, for example, from Valois, France (eg. DF10, DF30, DF60), Bespak pic, United Kingdom (eg. BK300, BK356, BK357) and 3M-Neotechnic Limited, United Kingdom (eg. Spraymiser™). The metering valves are used in association with commercially available canisters, for example metal canisters, such as aluminium canisters, suitable for delivering pharmaceutical aerosol formulations.

MDIs incorporating valve seals/gaskets as described above generally perform adequately with chlorofluorocarbon propellants (CFC's) such as propellant 11 (CCl₃F), propellant 114 (CF₂ClCF₂Cl) and propellant 12 (CCl₂F₂) or mixtures thereof. However, these propellants may now provoke the degradation of stratospheric ozone. Thus, there is a need to provide aerosol formulations for medicaments that employ so-called “ozone-friendly” propellants.

A class of propellants which may have minimal ozone-depleting effects (in comparison to conventional CFC propellants) include, but are not limited limited to, hydrofluoroalkanes (HFA's) particularly 1,1,1,2-tetrafluoroethane (HFA134a), 1,1,1,2,3,3,3-heptafluoro-n-propane (HFA 227) and mixtures thereof. However there have been problems associated with stabilising the pharmaceutical aerosol formulations prepared using the new class of HFA propellants.

Pharmaceutical aerosol formulations generally comprise a solution or a suspension. A mixture of suspension and some (perhaps trace) amount of dissolved medicament is also possible, but generally undesirable as discussed below. Some solution formulations suffer the disadvantage that the drug substance contained therein is more susceptible to degradation. Furthermore there may be problems associated with controlling the size of the droplets that influence the therapeutic profile. For this reason, suspensions are generally preferred.

In general, the efficiency of the aerosol device, such as an MDI, is a function of the dose deposited at the appropriate site in the lungs. Deposition may be affected by several factors, of which one of the most important is the aerodynamic particle size. Solid particles and/or droplets in an aerosol formulation can be characterised by their mass median aerodynamic diameter (MMAD, i.e., the diameter around which the mass aerodynamic diameters are distributed equally).

In suspension formulations, particle size generally is controlled during manufacture by the size to which the solid medicament is reduced, usually by emicronisation. However, where the suspended drug has a sufficient solubility in propellant, a process known as Ostwald Ripening can lead to particle size growth. Also, particles may have the tendency to aggregate, or adhere to parts of the MDI eg. canister or valve. Furthermore the drug may have the tendency to be absorbed into any untreated and/or uncoated rubber components of the valve, especially when stored for a prolonged period. In particular fine particles may be preferentially absorbed. The effect of Ostwald ripening and especially of drug deposition may be particularly severe for potent drugs (including salmeterol xinafoate) which are generally formulated in low doses.

Pharmaceutical aerosol formulations generally comprise a suspension of a medicament, one or more liquid propellants, optionally with a co-propellant, and optionally an adjuvant such as a solvent or a surfactant and/or other excipients. The aerosol formulation is under pressure in the canister.

The problems mentioned above have been addressed by the addition of one or more of adjuvants such as alcohols, alkanes, dimethyl ether, surfactants (e.g., including fluorinated and non-fluorinated surfactants, carboxylic acids, polyethoxylates, etc.) and even conventional chlorofluorocarbon propellants in small amounts intended to minimise potential ozone damage as disclosed in, for example, EP0372777, WO91/04011, WO91/11173, WO91/11495 and WO91/14422, which are incorporated herein by reference.

Excipient free formulations of salmeterol xinafoate are described in WO93/11743, which is incorporated herein by reference.

Furthermore, WO96/32345, WO96/32151, WO96/32150 and WO96/32099 (which are incorporated herein by reference) disclose aerosol canisters coated with one or more fluorocarbon polymers optionally in combination with one or more non-fluorocarbon polymers that reduce the deposition (on the canister walls) of drug particles of the pharmaceutical alternative propellant aerosol formulation contained therein.

It is advantageous that the prescribed dose of aerosol medication delivered from MDIs to the patient (1) consistently meet the specifications claimed by the manufacturer and (2) comply with the requirements of the FDA and other regulatory authorities. That is, it is advantageous that every dose delivered from the MDI be within close tolerances. Therefore, it is advantageous that the formulation be substantially homogeneous throughout the administered dose at the time of actuation of the metering valve. It is also advantageous that the concentration of the suspension does not vary significantly when stored for a prolonged period of time.

To obtain regulatory approval, pharmaceutical aerosol formulation products satisfy strict specifications. One parameter.(for which a specification is usually set) is the fine particle mass (FPM). FPM is a manner of evaluating the amount of drug substance that has the potential to reach the inner lungs (i.e., the small bronchioles and alveoli, based on the amount of drug particles with a diameter within a certain range, usually less than 5 microns).

The FPM of an actuation from an MDI is generally calculated based on the sum of the amount of drug substance deposited on stages 3, 4 and 5 of an Andersen Cascade Impaction stack as determined by standard HPLC analysis.

It is advantageous that the FPM of the pharmaceutical aerosol formulation for all the doses dispensed from the MDI are within the specification set, particularly after the MDI has been stored for a prolonged period of time.

It has been thought that the concentration of drug in the suspension (and thus the dose dispensed, of particulate salmeterol xinafoate and HFA formulations) may decrease over time with the adsorption of drug into the rubber components of the valve. This may be observed as a decrease in the Total Drug Content (TDC) of the can. This problem may be accelerated by the ingression of moisture into the formulation from the surrounding ambient conditions.

It is further thought that the FPM and mean dose of salmeterol xinafoate HFA 134a formulations decreases over time with the ingression of water into the formulation and/or deposition and/or absorption/adsorption.

Deposition of drug particles on other valve components, particularly the metering chamber may also contribute to the formulation stability problems observed such as inconsistencies in the doses dispensed, which become particularly acute over increasing numbers of actuations.

The problem with deposition may be particularly exacerbated when excipient-free aerosol formulations are employed based on the propellants 1,1,1,2-tetrafluoroethane (HFA 134a) and 1,1,1,2,3,3,3-heptafluoro-n-propane (HFA 227). It further is thought to increase with length of storage of the aerosol, particularly when stored at high temperature and/or high humidity.

The suspension concentration, dose and FPM of formulations of salmeterol xinafoate in suspension in a HFA propellant, obtained from an MDI are advantageously be stabilised by reducing the deposition on the valve component(s) and/or reducing the drug absorption into rubber components and/or effectively controlling the ingression of water into the formulation during storage and use by employing particular valve materials. The present invention also advantageously treats and coats the elastomeric components of an MDI to reduce friction which can cause sticking of and/or subsequent damage to such components during filling and use thereof.

The transition from MDI's utilizing CFC propellants to those using HFA propellants has presented a number of problems and challenges. In particular, drug-containing aerosol formulations containing one or more HFA propellants generally 20 require the presence of ethanol in the formulation. Conventional seals and gaskets swell in the presence of ethanol. As a result, conventional seals and gaskets that swell lose, at least in part, sealing ability. To improve sealing in the presence of ethanol, elastomeric (e.g. EPDM) seals and gaskets were employed because they are known to be less vulnerable to swelling. However, untreated elastomeric gaskets lack sufficient or optimal lubricity in many applications. Hence, the untreated EPDM seal or gasket can wear unevenly and lose sealing ability. Insufficient lubricity and wear can also result in sticking of the valve.

The present invention advantageously overcomes the problems in the prior art seals and gaskets utilized in MDI's by improving sealing and lubricity throughout the life of the inhaler, including filling of the MDI.

Another problem associated with conventional MDI seals and gaskets is that the bulk and surface generally include a plurality of different substances that function as contaminants/impurities and extractives. For example, conventional bulk elastomers contain fillers, additives, colorants, and curing agents. The surface of the sheet stock elastomer may be contaminated with a variety of silicones, soaps, lubricants, dirt, grease and other common contaminants. Such contaminants inhibit or prevent suitable adhesion of a coating or effectiveness of a surface treatment. The extractives are problematic in terms of detrimentally affecting the efficacy and safety of the aerosol drug formulation.

The present invention advantageously improves safety and efficacy by creating a barrier to extractables from the gasket and by forming a barrier to water vapor ingression in the surrounding environment.

Yet another problem associated with MDI seals and gaskets is that they tend to degrade overtime due to exposure to the propellant and any solvents in the drug formulation, particularly because the seals/gaskets are under high compression forces and pressures. Over time, plasticizers and fillers are extracted from the elastomeric seals and gaskets, which degrade the elastomer. Thus, the degraded seals and gaskets lose flexibility, lubricity and sealing ability.

The present invention prevents or substantially reduces degradation by adhering an organotitanium low friction barrier coating to the seal/gasket surface. The coated/treated seals and gaskets of the present invention advantageously improve the performance and longevity by chemically bonding a dry lubricant coating that is free of silicone and elemental silicon. Liquid silicone is conventionally used to coat various valve stem components. However, that process is multi-step and time consuming. The silicone process is also inefficient because it is logistically complex, particularly in the valve assembly process.

The pretreatment of the present invention permits superior adhesion and bonding of the organotitanium-based coating. The coated seals/gaskets also have a low coefficient of friction and high lubricity, which reduces mechanical wear of the seals/gaskets and reduces/eliminates valve sticking and drug particle deposition. The seal/gasket surface may also be hydrophobic or hydrophilic depending upon the organotitanium monomer employed. The organotitanium coating also advantageously functions as a superior barrier to gases, vapors (such as water vapor) and solvents, which is particularly important in HFA aerosol formulations that tend to destabilize upon water vapor ingress. The barrier may also reduce the level of extractives from the elastomer and seal off any mould release agents on the surface of the elastomer. The barrier functionality also advantageously reduces or prevents propellant leakage and egress. The coated seal/gasket is further chemically and thermally stable, flexible and chemically compatible with aerosol drug formulations.

Other advantages and benefits of the present invention are set forth below and in the included drawings.

### DESCRIPTION OF THE DRAWINGS

The present invention will become more fully understood from the detailed description provided herein and the accompanying drawings, which are provided by way of illustration only, and, thus, are not limiting of the present invention.

FIG. 1 shows a cross-sectional view of one embodiment of the metering valve of the present invention.

FIG. 2 shows another cross-sectional view of one embodiment of the metering valve of the present invention.

FIG. 3 shows a cross-sectional view of one embodiment of the ring and strough structure of the present invention.

### SUMMARY OF THE INVENTION

One aspect of the invention relates to a method of treating an elastomeric substrate comprising, in any suitable order, the acts of: providing an elastomeric substrate in a bath comprising an alcohol and an alkaline material at a bath temperature effective for treatment; providing ultrasonic energy at a treatment-effective frequency and power level to the bath for a time period sufficient to treat the elastomeric substrate; rinsing the treated elastomeric substrate with de-ionized water; and, drying the rinsed and treated elastomeric substrate.

Preferably, the bath comprises ethanol and potassium hydroxide. More preferably, the bath comprises 80-99 wt. % ethanol and 1-20 wt. % potassium hydroxide, and still more preferably about 91 wt. % ethanol and about 9 wt. % potassium hydroxide.

The ultrasonic energy is preferably provided to the bath for 10-20 minutes, and wherein the bath temperature is in the range of 40-60° C., and still more preferably for about 15 minutes, and wherein the bath temperature is about 50° C.

Preferably, the elastomeric substrate is made from an acrylonitrile butadiene or an ethylene propylene diene monomer. The elastomeric substrate may further include one or more fillers, additives, curing agents, and/or crosslinking agents. The rinsed and treated elastomeric substrate is-preferably dried in a convection oven.

Another aspect of the invention relates to a method of treating and coating an elastomeric substrate comprising, in any suitable order, the acts of: providing an elastomeric substrate in a bath comprising an alcohol and an alkaline material at a bath temperature effective for treatment; providing ultrasonic energy at a treatment-effective frequency and power level to the bath for a time period sufficient to treat the elastomeric substrate; rinsing the treated elastomeric substrate with de-ionized water; drying the rinsed and treated elastomeric substrate; providing a reactor chamber to contain the rinsed and treated elastomeric substrate; feeding an organic titanate into the reactor chamber maintained at a suitable pressure; applying a voltage to the reactor chamber generating a plasma; and, purging the reactor chamber.

The list of organic titanates that may be used in the present invention include, but are not limited to, tetraalkyl titanates and titanate chelates. Exemplary tetraalkyl titanates include, but are not limited to, tetraisopropyl titanate, tetra-n-butyl titanate and tetrakis(2-ethylhexyl)titanate. Exemplary titanate chelates include, but are not limited to, acetylacetonate titanate chelate, ethyl acetoacetate titanate chelate, triethanolamine titanate chelate, and, lactic acid titanate chelate ammonium salt. These organic titanates are available from DuPont's Performance Chemical line of TYZOR® organic titanate products. The organic titanate is preferably deposited onto the elastomeric substrate by means of utilizing Plasma Enhanced Chemical Vapor Deposition (PECVD) techniques. Other suitable vapor deposition processes may also be used to deposit the organic titanate.

An inert gas may also be fed into the reactor chamber maintained at a suitable pressure. The etching gas may be fed into the reactor chamber prior to the organic titanate generating an etched rinsed and treated elastomeric substrate. The etching gas may alternatively be fed into the reactor chamber with the organic titanate. Preferably, the ratio of the inert etching gas to organic titanate is in the range of 20:1 to 40:1, and more preferably in the range of 30:1 to 35:1, and still more preferably about 33⅓:1. The etching inert gas is preferably argon or oxygen. The reaction chamber is preferably purged with nitrogen. The organic titanate is preferably tetraisopropyl titanate.

Preferably, the pressure of the reaction chamber is maintained at about 50 milliTorr and the applied voltage may be about 250 volts. The plasma generated from the etching gas may react with the substrate for up to about 10 minutes. The plasma generated from the etching gas and the organic titanate may react with the substrate for up to about 10 minutes.

Another aspect of the invention is a seal for use in an inhaler comprising: an elastomeric substrate; and, a titanium-containing coating on at least a portion of the substrate having a film thickness.

Still another aspect of the invention is a gasket for use in an inhaler comprising: an elastomeric substrate; and, a titanium-containing coating on at least a portion of the substrate having a film thickness.

Still another aspect of the invention is a seal for use in an inhaler made by a method comprising, in any suitable order, the acts of providing an elastomeric substrate in a bath comprising an alcohol and an alkaline material at a bath temperature effective for treatment; providing ultrasonic energy at a treatment-effective frequency and power level to the bath for a time period sufficient to treat the elastomeric substrate; rinsing the treated elastomeric substrate with de-ionized water; drying the rinsed and treated elastomeric substrate; providing a reactor chamber to contain the rinsed and treated elastomeric substrate; feeding inert argon gas into the reactor chamber; applying a voltage to the reactor to create an agron plasma for etching; feeding an organic titanate into the reactor chamber maintained at a suitable pressure; applying a voltage to the reactor chamber generating a plasma; and, purging the reactor chamber.

Yet another aspect of the invention is a gasket for use in an inhaler made by a method comprising, in any suitable order, the acts of providing an elastorheric substrate in a bath comprising an alcohol and an alkaline material at a bath temperature effective for treatment; providing ultrasonic energy at a treatment-effective frequency and power level to the bath for a time period sufficient to treat the elastomeric substrate; rinsing the treated elastomeric substrate with de-ionized water; drying the rinsed and treated elastomeric substrate; providing a reactor chamber to contain the rinsed and treated elastomeric substrate; feeding an organic titanate into the reactor chamber maintained at a suitable pressure; applying a voltage to the reactor chamber generating a plasma; and, purging the reactor chamber.

Still another aspect of the invention relates to a metering valve comprising: a valve body; a metering chamber; a valve stem; and, one or more stem seals comprising an elastomeric substrate and a titanium-containing coating on at least a portion of the substrate having a film thickness, wherein the metering valve is suitable for metering a drug suspension comprising a medicament and a liquid propellant, and wherein the medicament is a member selected from the group consisting of salmeterol, salbutamol, formoterol, ipratroprium, fluticasone, beclomethasone, budesonide, terbutaline, salts esters, solvates thereof, and combinations thereof.

Another aspect of the invention relates to a metered dose inhaler comprising: a cannister in communication with a metering valve, the metering valve comprising: a valve body; a metering chamber; a valve stem; and, one or more stem seals comprising an elastomeric substrate and a titanium-containing coating on at least a portion of the substrate having a film thickness, wherein the metering valve is suitable for metering a drug suspension comprising a medicament and a liquid propellant, and wherein the medicament is a member selected from the group consisting of salmeterol, salbutamol, formoterol, ipratroprium, fluticasone, beclomethasone, budesonide, terbutaline, salts esters, solvates thereof, and combinations thereof.

Another aspect of the invention relates to a drug product comprising: a cannister containing a drug suspension comprising a propellant and a medicament in communication with a metering valve, the metering valve comprising: a valve body; a metering chamber; a valve stem; and, one or more stem seals comprising an elastomeric substrate and a titanium-containing coating on at least a portion of the substrate having a film thickness, wherein the metering valve is suitable for metering the drug suspension, and, wherein the medicament is a member selected from the group consisting of salmeterol, salbutamol, formoterol, ipratroprium, fluticasone, beclomethasone, budesonide, terbutaline, salts esters, solvates thereof, and combinations thereof.

### DETAILED DESCRIPTION OF THE PREFERRED EMBODIMENTS

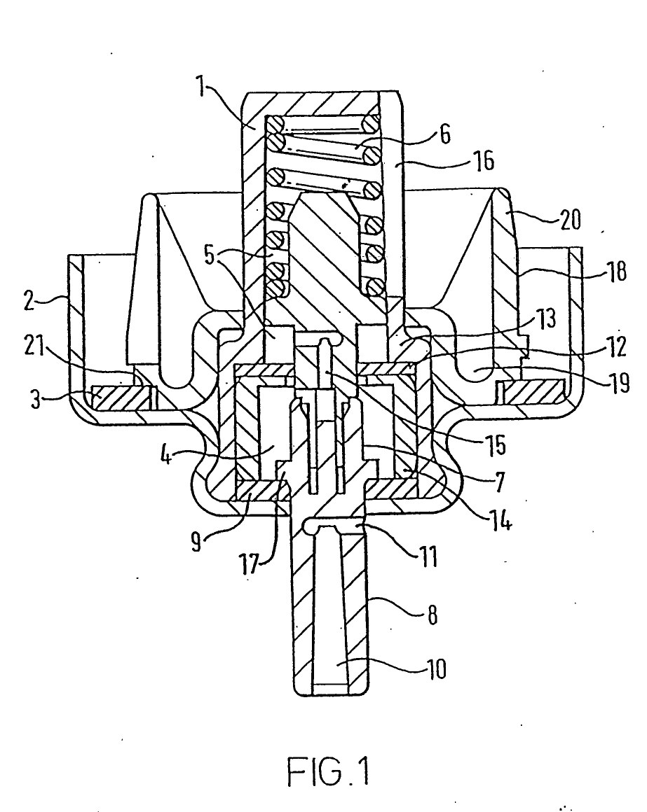

The valve according to a first embodiment of the invention as shown in FIG. 1 comprises a valve body I sealed in a ferrule 2 by means of crimping, the ferrule itself being set on the neck of a container (not shown) with the interposition of a gasket 3 in a well-known manner. The container is filled with a suspension of a medicament in liquid propellant HFA134a.

Medicaments suitable for this purpose include, but are not limited to, medicaments for the treatment of respiratory disorders, such as asthma, bronchitis, chronic obstructive pulmonary diseases and chest infections. Other medicaments may be employed having efficacy for inhalation therapy and which may be formulated as a suspension. Suitable medicaments include, but are not limited to, analgesics, e.g. codeine, dihydromorphine, ergotamine, fentanyl or morphine; anginal preparations, e.g. diltiazem; antiallergics, e.g. cromoglycate, ketotifen or neodocromil; antiinfectives e.g. cephalosporins, penicillins, streptomycin, sulphonamides, tetracyclines and pentamidine; antihistamines, e.g. methapyrilene; anti-inflammatories e.g. fluticasone propionate, beclomethasone dipropionate, flunisolide, budesonide, or triamcinolone acetonide; antitussives, e.g. noscapine; bronchodilators, e.g. salmeterol, salbutamol, ephedrine, adrenaline, fenoterol, formoterol, isoprenaline, metaproterenol, phenylephrine, phenylpropanolamine, pirbuterol, reproterol, rimiterol, terbutaline, isoetharine, tulobuterol orciprenaline, or(−)4-amino-3,5-dichloro-α-[[[6-[2-(2-pyridinyl)ethoxy]-hexyl]amino]methyl]benzenemethanol; diuretics, e.g. amiloride; anticholinergics e.g. ipratropium, atropine or oxitropium; hormones, e,g, cortisone, hydrocortisone or prednisolone; xanthines e.g. aminophylline, choline theophyllinate, lysine theophyllinate or theophylline and therapeutic proteins and peptides, e.g. insulin or glucagon, and combinations thereof. It will be clear to a person skilled in the art that, where appropriate, the medicaments may be used in the form of salts (e.g. as alkali metal or amine salts or as acid addition salts) or as esters (e.g. lower alkyl esters) or as solvates (e.g. hydrates) to optimise the activity and/or stability of the medicament. Preferred medicaments are salbutamol, salbutamol sulphate, salmeterol, salmeterol xinafoate, fluticasone propionate, beclomethasone dipropionate, terbutaline sulphate, ipratropium bromide, and combinations thereof. It is to be understood that the suspension of medicament may consist purely of one or more active ingredients.

The valve body 1 includes a metering chamber 4 at the lower portion of the valve and a sampling chamber 5 at the upper portion. The sampling chamber 5 also functions as a housing for a return spring 6. The words “upper” and “lower” are used to indicate orientation with the neck of the container and valve oriented in the lower position as shown in FIG. 1. The valve body 1 includes a valve stem 7 including an external stem portion 8 extending outside the valve through a lower stem seal 9 and ferrule 2. The external stem portion 8 includes an axial or longitudinal canal 10 opening at the outer end of the stem 7 and in communication with a radial passage 11.

The internal portion of stem 7 has a diameter such that it can pass slideably through an opening in an upper stem seal 12. The stem 7 also engages the periphery of upper stem seal 12 sufficiently to provide a seal. Upper stem seal 12 is held in position against a step 13 formed in the valve body 1 between the lower and upper parts by a sleeve 14. The sleeve 14 defines the metering chamber 4 between lower stem seal 9 and upper stem seal 12. The valve stem 7 includes a passage 15 which (when the stem is in the inoperative positive shown in FIG. 1) provides a communication between the metering chamber 4 and sampling chamber 5. The valve stem 7 also communicates with the interior of the container via slots 16 formed in the side of the valve body 1. The slots 16 may be arranged equiangular around the valve body 1 extending in an axial direction with respect thereto. Each slot 16 may have a width of approximately 1 mm and a length slightly less than the length of the sampling chamber 5 so that the suspension within the container can flow freely through the entire sampling chamber 5.

Valve stem 7 is biased downwardly by return spring 6 and is provided with a shoulder 17 that abuts lower stem seal 9. In the position as shown in FIG. 1, shoulder 17 abuts against lower stem seal 9. Radial passage 11 opens below lower stem seal 9 isolating metering chamber 4 from canal 10. The suspension in the container is suitable sealed from the surrounding environment.

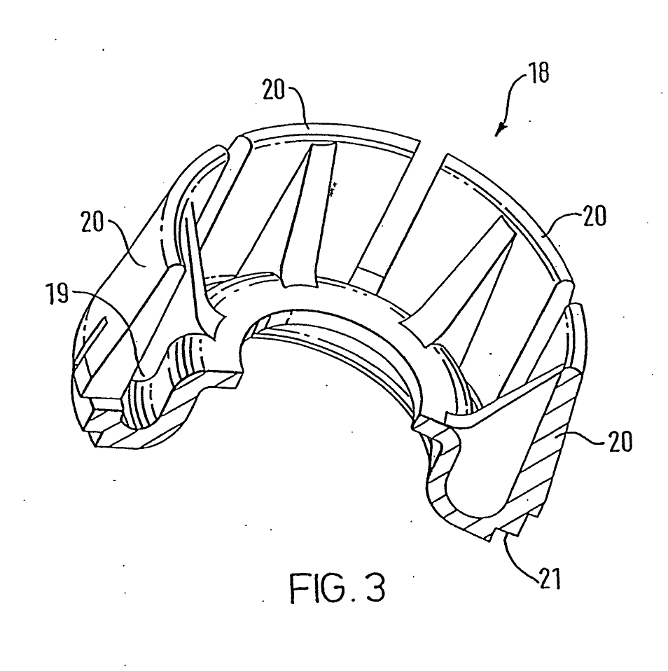

A ring 18 is disposed around the valve body below the slots. The ring 18 is formed with a number of portions of reduced axial thickness giving a “U” shaped cross section extending in a radial direction. A number of troughs 19 are formed around the valve body. As shown in FIGS. 1 and 3, the ring is formed as a separate component, however it may be integrally formed with one or more other valve components. Preferably, the ring 18 is moulded from nylon or other suitable moulding materials. Preferably, the ring 18 has an inner annular contacting rim of a diameter suitable to provide a friction fit over the upper part of valve body 1. The ring 18 seats against a step 13 below the slots 16. Alternatively, the ring 18 may be formed as an integrally moulded part of valve body 1.

As shown in FIG. 3, the outer wall of the ring extends axially forming a number of equiangular-spaced slots creating vanes 20 extending upwards from the lower part of the ring. Preferably, six slots and six vanes are employed. Alternatively, more or fewer slots and vanes may be employed. The lower part of the ring includes a seat 21 for gasket 3 to correctly position the gasket during assembly. The seat 21 also allows increasing the inner diameter of the gasket to reduce the size and area of contact of the gasket. The seat/gasket structure advantageously prevents or reduces the possibility of impurities being leached out of the gasket into the drug suspension.

The container is shaken to homogenise the drug suspension. The suspension flows freely through the slots 16 in the sampling chamber 5 ensuring that the suspension in the sampling chamber is thoroughly mixed with the suspension in the container. The flow of suspension also disperses or re-suspends drug particles that agglomerates within the sampling chamber 5. The suspension also flows around the vanes 20 causing turbulence and a swirling motion of the suspension dispersing drug particle sediment or agglomerate near the ring.

To actuate the valve, the valve stem 7 is depressed against the force of the spring 6. When the valve stem is depressed, both ends of the passage 15 lie on the side of upper stem seal 12 remote from the metering chamber 4. A dose of drug formulation is metered within the metering chamber. The valve stem 7 may be further depressed moving the radial passage 11 into the metering chamber 4 as the upper stem seal 12 seals against the valve stem body. The metered dose exits through the radial passage 11 and the outlet canal 10.

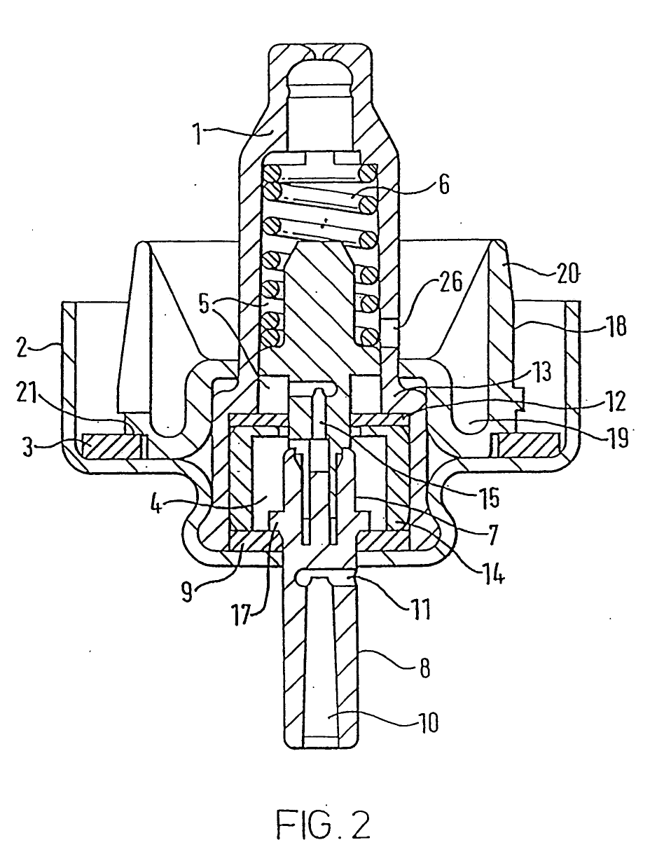

Releasing the valve stem causes the spring 6 to return the stem 7 to the position shown in FIGS. 1 and 2. Then, the passage 15 again communicates between the metering chamber 4 and the sampling chamber 5. At this position, liquid passes under pressure from the container through slots 16 and passage 15 and into the metering chamber 4.

In the actuable orientation of the container and valve, the “U” shaped configuration of the ring 18 around the valve body provides a trough 19 that lies below the slots 16. The trough serves to accommodate drug particle sediment/agglomerate that is not re-dispersed in suspension. The trough 19 also ensures that the suspension (entering the sampling chamber 5 through the slots 16) is a homogenous suspension free of drug particle sediment and agglomerate. The ring 18 further also reduces the volume of suspension that can be accommodated within the container below the slots 16.

The coated/treated elastomeric gaskets and seals of the present invention advantageously improve sealing and lubrication compared to untreated elastomeric seals and gaskets. In particular, drug-containing aerosol formulations containing one or more HFA propellants generally require the presence of ethanol in the formulation. Conventional seals and gaskets swell in the presence of ethanol. As a result, conventional seals and gaskets that swell lose, at least in part, their sealing ability. To improve sealing in the presence of ethanol, elastomeric (e.g. EPDM) seals and gaskets were employed because they are known to be less vulnerable to swelling. However, untreated elastomeric gaskets lack sufficient or optimal lubricity in many applications. Hence, the untreated EPDM seal or gasket can wear unevenly and lose sealing ability. Insufficient lubricity and wear can also result in sticking of the valve.

In the foregoing, the expressions “polymer of EPDM” and “EPDM polymer” are used interchangeably. The preferred liquefied propellant gas is 1,1,1,2-tetrafluoroethane. EPDM polymer is available from a variety of suppliers including West and Parker Seals.(USA). A gasket/seal constructed from a polymer of EPDM includes greater than 90 wt. % of EPDM polymer, preferably greater than 95% of EPDM polymer, and more preferably greater than 99% of EPDM polymer. In one embodiment of the invention, an MDI valve is sealed to the canister by means of a can sealing gasket 3 which is substantially constructed from a polymer of EPDM. Preferably the valve is a metering valve, but other types of valves suitable for a pharmaceutical inhalation device may also be used in the present invention.

EPDM is a member of a broad class of Thermoplastic Elastomers (TPE's) which is also referred to as Thermoplastic Rubbers (TPR's) and Thermoplastic Vulcanizates (TPV's). Nitrile rubbers may also be used to make seals and gaskets for use in MDI's, and include, for example, polybutadiene copolymerized with acrylonitrile. Exemplary members of these classes of elastomers and rubbers include, but are of course not limited to, SANTOPRENE®, VYRAM®, VISTAFLEX®, DYTRON®, GEOLAST®, and TREFSIN®. While rubber gaskets and seals containing extractives can be problematic, they generally contain appropriate amounts of conventional fillers, additives and curing agents.

In another embodiment of the present invention, the metering valve includes a metering chamber 4 defined by a liner 14 and the upper 12 and a lower 9 sealing gaskets through which pass a valve stem 8. The valve is sealed to the canister with the sealing gasket 3.

The said metering chamber may include the liner 14. The liner may be treated and/or coated. The line 14 may be constructed from of any material with suitable characteristics. For example, the liner 14 may be moulded from nylon, polybutylene terephthalate PBT (polyester), acetal (polyoxymethylene) and tetrabutyrene terephthalate (TBT). The liner 14 may also be made from a metallic material compatible with the drug formulation, such as stainless steel or aluminium.

The invention also includes the surface of the metering chamber 4 being substantially fluorinated. A fluorinated film surface on the metering chamber 4 advantageously reduces drug deposition on the metering chamber, particularly when used in conjunction with a drug suspension formulation comprising salmeterol xinafoate and HFA propellant.

In particular, the surface treatment of the walls of the metering chamber 4 include using one or more fluorine-containing plasma comprising fluorinated small molecule monomers (or precursors) such as C₁₋₁₀ perfluoroalkanes, including but not limited to, perfluorocycloalkanes; C₂₋₁₀ perfluoroalkenes; fluoroalkanes including fluorocycloalkanes or fluoroalkenes wherein a substantial proportion of the hydrogens are replaced with fluorine or mixtures of fluorine-containing molecules thereof. Furthermore, the fluorine-containing plasma may be made by employing, in combination with the fluorinated precursors, one or more non-fluorocarbon compounds. More preferably, the plasma is made from C₁₋₁₀perfluoroalkanes precursors.

The fluorine-containing plasma coating is used to apply a fluorinated polymer on at least a portion of any surface any of the various valve components, such as the metering chamber, the valve stemm the ring, the trough, the liner (if employed), the sampling chamber, etc. Polymerisation or direct modification of the component surface is accomplished by interchanging hydrogen ions in the substrate with fluorine ions. The cold plasma fluorination process may be performed under vacuum at ambient temperature. The components to be coated are placed inside a reactor chamber which has been purged and evacuated. The fluorine monomer (or precursor) is fed into the reactor chamber at a controlled rate. The plasma is ignited within the chamber, creating the plasma, and maintained for a predetermined period of time and at a chosen power setting. At the end of the treatment cycle, the plasma is extinguished, the chamber flushed (or purged, such as with nitrogen) and the products retrieved. A thin layer (e.g., 1-10 microns) of fluorine-containing polymer is bonded to the surface of the valve component.

For plasma polymerization typically temperatures in the range of about 20° C. to about 100° C. may be employed. The surface of the component especially the metering chamber may require activating in order to facilitate more effective coating of the surface by improving the adhesion of the coating to the surface.

Preferably the components to be plasma coated with the fluorine-containing plasma will be pre-treated to remove any surface contamination and/or to activate the surface. This may be achieved by for example treatment of the components with an etching gas such as oxygen or argon. In the process radicals react with the plastic or metal substrate e.g the component is exposed to a low pressure oxygen plasma environment which creates polar groups on the components surface which are more conducive to bonding with plasma coating to be applied.

Alternatively the metering chamber (especially when composed of a plastics material for example those described above) may be surface treated with a siloxane-containing plasma such as dimethyl siloxane using a similar process as that described above for fluoroplasma coating.

Alternatively metering chambers (especially when composed of a metallic material) can be coated by conventional techniques using fluorocarbon polymers which include fluorocarbon polymers which are made of multiples of one or more of the following monomeric units: tetrafluoroethylene (PTFE), fluorinated ethylene propylene (FEP), perfluoroalkoxyalkane (PFA), ethylene tetrafluoroethylene (ETFE), vinyldienefluoride (PVDF), and chlorinated ethylene tetrafluoroethylene. Fluorinated polymers, which have a relatively high ratio of fluorine to carbon, such as perfluorocarbon polymers, e.g., PTFE, PFA and FEP may be preferable.

The fluorinated polymer is optionally blended with non-fluorinated polymers such as polyamides, polyimides, polyamideimides, polyethersulfones, polyphenylene sulfides, and amine-formaldehyde thermosetting resins. These added polymers often improve adhesion of the polymer coating to the substrate. Preferred polymer blends are PTFE/FEP/polyamideimide, PTFE/polyether sulphone (PES) and FEP-benzoguanamine. The most preferred polymer coating is a blend of PTFE and PES. A coating of pure FEP is also of considerable interest.

A technique for applying such coatings to for example, a metal (such as aluminium or stainless steel) is where the metal is precoated as coil stock and cured before being stamped or drawn into the can shape. This method is well suited to high volume production for two reasons. First, the art of coating coil stock is well developed and several manufacturers can custom coat metal coil stock to high standards of uniformity and in a wide range of thicknesses. Second, the precoated stock can be stamped or drawn at high speeds and precision by essentially the same methods used to draw or stamp uncoated stock.

Other techniques for coating techniques includes electrostatic dry powder coating or by spraying preformed MDI components with formulations of the coating fluorinated polymer/polymer blend and then curing. The preformed MDI components may also be dipped in the fluorocarbon polymer/polymer blend coating formulation and cured, thus becoming coated on the inside and out. The fluorocarbon polymer/polymer blend formulation may also be poured inside the MDI components then drained out leaving the insides with the polymer coat.

The appropriate curing temperature is dependent on the polymer blend chosen for the coating and the coating method employed. However, for coil coating and spray coating temperatures in excess of the melting point of the polymer are typically required, for example, about 50° C. above the melting point for up to about 20 minutes such as about 5 to 10 minutes e.g. about 8 minutes or as required. For the above named preferred and particularly preferred polymer blends curing temperatures in the range of about 300° C. to about 400° C, e.g. about 350° C. to 380° C. are suitable.

Where the components are coated, and subsequently cured, the substrate components may be prepared from strengthened materials to ensure they withstand the process. Thus, an aspect of the invention is a process for preparing a container as described above, wherein the surface treatment of the metering chamber comprises a process for applying a coating of a fluorocarbon polymer optionally in combination with a non-fluorocarbon polymer.

Conversely alternative polymer coatings may be used on the components which may be dipped or bath immersed into a treatment tank containing a solution of polymeric compound. Usually the components are immersed in the solution at room temperature for at least one hour, for example, 12 hours, thus being treated both internally and externally.

The treated components are preferably washed with solvent and dried at an elevated temperature for example 50-100° C. optionally under vacuum. Examples of suitable coating materials include of fluoropolyethers having functionalised ends groups with a general formula R_{f}—O(C₃F₆O)ₘ(CFX)ₙ—CFX—Y-Zₚ as described in U.S. Pat. No. 4,746,550 (incorporated herein by reference) including perfluoropolyethers having functional groups capable of anchoring the coating to the substrate such as carboxyl, ester, amide, hydroxyl, isocyanate, epoxy, silane for example —CONR²R³ wherein R² and R³ may be independently selected from amongst other things hydrogen, or a silyl ether (e.g. SiRₜ(OR)₃₋ₜ or a fluoropolyether having hydroxylic functionality of the type —CF₂CH₂OH, —CF₂CFXCH₂OH (wherein X is Cl or F) or —CF(CF₃)CH₂OH as described in U.S. Pat. No. 6,071,564 (incorporated herein by reference); phosphoric diesters of formula [XCF₂CF₂O(CFXCF₂O)XCFXCH₂O]₂PO(OM) as described in U.S. Pat. No. 3,492,374 (incorporated herein by reference) or phosphoric mono-ester of formula [R_{f}—O—CFY—L—O]ₘP═O(O⁻Z⁺)₃₋, as described in EP 0 687 533 (incorporated herein by reference) wherein L is a divalent organic group; m=1; Y is —F or —CF₃; Z⁺ is selected from H⁺, M⁺ where M is an alkali metal; N(R)₄⁺ where the R groups independently represent H or C₁₋₆alkyl; R_{f} is a polyperfluoroalkyleneoxide chain.

The fluoropolyethers described above may be used in combination with monofunctional fluoropolyethers having —CH₂OH terminals directly linked to a perfluoroalkyl group —CF₂, —CF₂CFX (wherein X is Cl or F) or CF(CF₃) optionally through a bridging group (CH₂CH₂)_{q} wherein q represents an integer from 1 to 6.

Other suitable coating materials also include polymeric compounds that are silane derivatives of perfluoropolyoxyalkanes with a molecular weight in the range 1600-1750 and those of the general formula: R¹—(CH₂)ᵥ—CF₂O—(C₂F₄O)ₓ—(CF₂O)_{y}CF₂—(CH₂)_{w}—R¹ (I) wherein R¹ comprises —(OCH₂—CH₂)_{Z}—OPO(OH)₂, wherein x, y and z are such that the molecular weight of the compound is 900-2100 and v and w independently represent 1 or 2.

Alternatively the metering chamber presents a substantially fluorinated surface to the formulation by virtue of being composed of a suitable substantially fluorinated material. Suitable fluorinated materials include fluorinated polymer/copolymer or mixtures thereof or a mixture of the fluorinated polymer in combination with non-fluorinated polymers conventionally used in the manufacture of valves, such as acetal, polyester (PBT). Examples of suitable fluorinated polymers include polytetrafluoroethylene (PTFE), ethylenetetrafluoroethylene (ETFE), polyvinyidienefluoride (PVDF), perfluoroalkoxyalkane (PFA), polyvinylfluoride (PVF), polychlorotrifluoroethylene (PCTFE), fluorinated ethylenepropylene (FEP) etc. Suitable copolymers include copolymers of tetrafluoroethylene (TFE) with PFA, TFE with hexafluoropropylene (HFP) (available as FEP 6107 and FEP 100 from DYNEON), VDF with HFP (commercially available as Viton A), TFE with perfluoro(propyl vinyl ether) (available as PFA 6515N from DYNEON), a blend of TFE, hexafluoropropylene and vinylidene fluoride (available commercially as THV 200G from DYNEON), etc.

It should be noted, however, that any conventionally available polymer, copolymer or mixture thereof which comprises a fluorinated polymer and which can be used to make the valve for use in an inhaler according to the invention may be suitable. Examples of mixtures of polymers and/or copolymers comprise, for example, up to 80% by weight fluorinated polymer, optionally up to 40% by weight fluorinated polymer, optionally up to 20% by weight fluorinated polymer or optionally up to 5% by weight of fluorinated polymer. Preferably, fluorinated polymers selected from PTFE, PVF and PCTFE are used as mixtures with non-fluorinated polymers. For example a suitable material is HOSTAFORM X329™ (Hoechst) which is a 5% PTFE/Acetal blend, HOSTAFORM C9021TF which is a 20% PTFE/Acetal blend, PTFE/PBT blends (for example, LNP WL4040), PTFE/PBT/silicone blends (for example, LNP WL4540).

Other suitable materials which the metering chamber may be constructed from includes one or more materials selected from the group consisting of a polyethylenetetrafluoroethylene, a polyvinyl-dienefluoride, a polyperfluoroalkoxyalkane, a polychlorotrifluoroethylene, a fluorinated polyethylene propylene, a copolymer of a polytetrafluoroethylene and a polyperfluoroalkoxyalkane, a copolymer of a polytetrafluoroethylene and a polyhexafluoropropylene, a copolymer of a polyvinyldienefluoride and a polyhexafluoropropylene, a copolymer of a polytetrafluoroethylene and a polyperfluoro(propyl vinyl ether); a blend of a polytetrafluoroethylene, a polyhexafluoropropylene a polyvinylidene fluoride, blends thereof and combinations thereof

The fluorinated polymers and mixtures thereof used in the invention can be moulded in any conventional manner, for example, by injection moulding, plastic moulding etc. The invention also relates to a container as described above wherein the valve stem presents a substantially fluorinated surface to the formulation.

Preferably the substantially fluorinated surface will result from surface treatment of the stem. Most preferably the surface treatment will comprise a process of plasma coating with highly fluorinated small molecules such as: C₁₋₁₀perfluoroalkanes including fluorocycloalkanes; C₂₋₁₀perfluoroalkenes; fluoroalkanes including fluorocycloalkanes or fluoroalkenes wherein a high proportion of the hydrogens have been replaced by fluorines or mixtures thereof as described above. Stems to be plasma coated with a fluorine-containing plasma may optionally be pretreated to remove surface contamination and/or activate the surface.

Alternatively stems may be coated by conventional techniques using fluorocarbon polymers optionally in combination with non-fluorocarbon polymer wherein the said coating requires curing after application as described above.

Additionally stems may be coated by processes using fluorocarbon polymers that require drying at temperatures between 50-100° C. as described above for metering chambers.

Alternatively the stem presents a substantially fluorinated surface to the formulation by virtue of being composed of a suitable fluorinated material.

Analogous processes and materials described above for metering chambers are suitable for the preparation of valve stems according to the invention.

The aerosol drug formulation includes a propellant, such as a C₁₋₄ hydrofluoroalkane, and preferably 1,1,1,2-tetrafluoroethane or 1,1,1,2,3,3,3-n-heptafluoropropane or a mixture thereof. Preferred formulations are free or substantially-free of excipients.

The drug formulations of the invention are preferably free of components that provoke the degradation of stratospheric ozone. In particular, the invention advantageously excludes chlorofluorocarbons such as CCl₃F, CCl₂F₂ and CF₃CCl₃. The invention is also advantageously substantially-free of any volatile adjuvant or surfactant, such as saturated hydrocarbons like propane, n-butane, isobutane, pentane, isopentane or a dialkyl ether like dimethyl ether. “Substantially free” will generally be understood to mean containing less than 0.01% w/w especially less than 0.0001% based on weight of medicament.

The drug formulation of the present invention is also substantially-free of polar adjuvants, such as C₂₋₆aliphatic alcohols and polyols like ethanol, isopropanol propylene glycol, glycerol and mixtures thereof.

Preferably, the particle size of the particulate (e.g. micronised) medicament is sufficient to permit inhalation of substantially all of the medicament into the lungs. More preferably, the particle size is less than 100 microns, more preferably less than 20 microns, still more preferably in the range 1-10 microns, and still more preferably 1-5 microns.

The concentration of medicament in the formulation may be in the range of 0.01-2% (all percentages are in terms of weight percent unless stated otherwise), more preferably 0.01-1%, and still more preferably 0.03-0.25% w/w. Drug formulations including salmeterol xinafoate may have a concentration of 0.03-0.15% w/w.

The term “metered dose inhaler” or MDI means a unit comprising a can, a secured cap covering the can and a formulation metering valve situated in the cap. MDI system includes a suitable channelling device. Suitable channelling devices comprise for example a valve actuator and a cylindrical or cone-like passage through which medicament may be delivered from the filled canister via the metering valve to the nose or mouth of a patient e.g. a mouthpiece actuator.

MDI canisters generally comprise a container capable of withstanding the vapor pressure of the propellant. A plastic, plastic-coated glass bottle, or, preferably, a metal can may be used. Preferably, the metal can is aluminium or an alloy thereof, which may be anodised, lacquer-coated and/or plastic-coated.

The cap may be secured onto the canister via welding such as ultrasonic welding or laser welding, screw fitting or crimping. MDIs taught herein may be prepared by methods of the art (e.g., see Byron, above and WO/96/32150). Preferably the canister is fitted with a cap assembly, wherein a formulation metering valve is situated in the cap. The cap is crimped in place.

A further aspect of the invention is a sealed container (as described above) capable of withstanding the pressure required to maintain the propellant as a liquid containing therein the aerosol formulation. Preferably the canister is composed of aluminium. Preferably the canister also presents a substantially fluorinated surface to the formulation. Preferably the canister is surface treated so as to present a substantially fluorinated surface to the formulation contained therein. More preferably the canister is surface treated by coating with a fluorocarbon polymer optionally in combination with a non-fluorocarbon polymer.

Fluorocarbon polymers selected from FEP and PTFE are particularly preferred for the surface treatment of canisters. FEP is especially preferred. A polymer blend of PTFE and PES is also especially preferred.

The surface treatment of the canister may be performed by methods analogous to those described above for valve components.

The formulations of the invention may be prepared by dispersal of the medicament in the selected propellant in an appropriate container, e.g. with the aid of sonication or a high-shear mixer. The process is desirably carried out under controlled humidity conditions.

The chemical and physical stability and the pharmaceutical acceptability of the aerosol formulations according to the invention may be determined by techniques well known to those skilled in the art. Thus, for example, the chemical stability of the components may be determined by HPLC assay, for example, after prolonged storage of the product. Physical stability data may be gained from other conventional analytical techniques such as, for example, by leak testing, by valve delivery assay (average shot weights per actuation), by dose reproducibility assay (active ingredient per actuation) and spray distribution analysis.

The suspension stability of the aerosol formulations according to the invention may be measured by conventional techniques, for example by measuring flocculation size distribution using a back light scattering instrument or by measuring particle size distribution by cascade impaction or by the “twin impinger” analytical process. As used herein reference to the “twin impinger” assay means “Determination of the deposition of the emitted dose in pressurised inhalations using apparatus A” as defined in British Pharmacopaeia 1988, pages A204-207, Appendix XVII C. Such techniques enable the “respirable fraction” of the aerosol formulations to be calculated. One method used to calculate the “respirable fraction” is by reference to “fine particle fraction” which is the amount of active ingredient collected in the lower impingement chamber per actuation expressed as a percentage of the total amount of active ingredient delivered per actuation using the twin impinger method described above.

Conventional bulk manufacturing methods and machinery well known to those skilled in the art of pharmaceutical aerosol manufacture may be employed for the preparation of large scale batches for the commercial production of filled canisters. Thus, for example, in one bulk manufacturing method a metering valve is crimped onto an aluminium can to form an empty canister. The particulate medicament is added to a charge vessel and liquefied propellant is pressure filled through the charge vessel into a manufacturing vessel, together with liquefied propellant containing the surfactant. The drug suspension is mixed before recirculation to a filling machine and an aliquot of the drug suspension is then filled through the metering valve into the canister.

In an alternative process, an aliquot of the liquefied formulation is added to an open canister under conditions which are sufficiently cold such that the formulation does not vaporise, and then a metering valve crimped onto the canister.

Typically, in batches prepared for pharmaceutical use, each filled canister is check-weighed, coded with a batch number and packed into a tray for storage before release testing.

Each filled canister is conveniently fitted into a suitable channelling device, prior to use, to form a metered dose inhaler system for administration of the medicament into the lungs or nasal cavity of a patient. Metered dose inhalers are designed to deliver a fixed unit dosage of medicament per actuation or “puff”, for example in the range of 10 to 5000 micrograms of medicament per puff.

Administration of medicament may be indicated for the treatment of mild, moderate, severe acute or chronic symptoms or for prophylactic treatment. It will be appreciated that the precise dose administered will depend on the age and condition of the patient, the particular particulate medicament used and the frequency of administration and will ultimately be at the discretion of the attendant physician. When combinations of medicaments are employed the dose of each component of the combination will in general be that employed for each component when used alone. Typically, administration may be one or more times, for example from 1 to 8 times per day, giving for example 1, 2, 3 or 4 puffs each time.

Suitable daily doses, may be, for example in the range 50 to 200 micrograms of salmeterol, depending on the severity of the disease.

Thus, for example, each valve actuation may deliver 25 micrograms of salmeterol (as xinafoate). Typically each filled canister for use in a metered dose inhaler system contains 60, 100, 120, 160 or 240 metered doses or puffs of medicament. An appropriate dosing regime for other medicaments will be known or readily available to persons skilled in the art.

The invention will now be described further with reference the following Example which serve to illustrate the invention but is not intended to be limiting.

1. A method of treating an elastomeric substrate comprising, in any suitable order, the acts of:
providing an elastomeric substrate in a bath comprising an alcohol and an alkaline material at a bath temperature effective for treatment;
providing ultrasonic energy at a treatment-effective frequency and power level to the bath for a time period sufficient to treat the elastomeric substrate;
rinsing the treated elastomeric substrate with de-ionized water; and,
drying the rinsed and treated elastomeric substrate.

2. The method of claim 1, wherein the bath comprises ethanol and potassium hydroxide.

3. The method of claim 2, wherein the bath comprises 80-99 wt. % ethanol and 1-20 wt. % potassium hydroxide.

4. The method of claim 3, wherein the bath comprises about 91 wt. % ethanol and about 9 wt. % potassium hydroxide.

5. The method of claim 1, wherein the ultrasonic energy is provided to the bath for 10-20 minutes, and wherein the bath temperature is in the range of 40-60° C.

6. The method of claim 5, wherein the ultrasonic energy is provided to the bath for about 15 minutes, and wherein the bath temperature is about 50° C.

7. The method of claim 1, wherein the elastomeric substrate is made from an acrylonitrile butadiene or an ethylene propylene diene monomer.

8. The method of claim 7, wherein the elastomeric substrate further comprises one or more fillers.

9. The method of claim 8, wherein the elastomeric substrate further comprises one or more crosslinking agents.

10. The method of claim 1, wherein the rinsed and treated elastomeric substrate is dried in a convection oven.

11. A method of treating and coating an elastomeric substrate comprising, in any suitable order, the acts of
providing an elastomeric substrate in a bath comprising an alcohol and an alkaline material at a bath temperature effective for treatment;
providing ultrasonic energy at a treatment-effective frequency and power level to the bath for a time period sufficient to treat the elastomeric substrate;
rinsing the treated elastomeric substrate with de-ionized water;
drying the rinsed and treated elastomeric substrate;
providing a reactor chamber to contain the rinsed and treated elastomeric substrate;
feeding inert gas into the reactor chamber;
applying a voltage to the reactor to create a first plasma;
feeding an organic titanate into the reactor chamber maintained at a suitable pressure;
applying a voltage to the reactor chamber generating a second plasma; and,
purging the reactor chamber.

12. The method of claim 11 further comprising:
feeding an etching gas into the reactor chamber maintained at a suitable pressure.

13. The method of claim 12, wherein the inert gas is fed into the reactor chamber prior to the organic titanate generating an etched rinsed and treated elastomeric substrate.

14. The method of claim 12, wherein the inert gas is fed into the reactor chamber with the organic titanate.

15. The method of claim 14, wherein the ratio of the inert gas to organic titanate is in the range of 20:1 to 40:1.

16. The method of claim 15, wherein the ratio of inert gas to organic titanate is in the range of 30:1 to 35:1.

17. The method of claim 16, wherein the ratio of inert gas to organic titanate is about 33⅓:1.

18. The method of claim 12, wherein the inert gas is argon or oxygen.

19. The method of claim 11, wherein the reaction chamber is purged with nitrogen.

20. The method of claim 11, wherein the organic titanate is tetraisopropyl titanate.

21. The method of claim 11, wherein the pressure of the reaction chamber is maintained at about 50 milliTorr.

22. The method of claim 11, wherein the applied voltage is about 250 volts.

23. The method of claim 12, wherein the plasma generated from the inert gas reacts with the substrate for up to about 10 minutes.

24. The method of claim 12, wherein the plasma generated from the inert gas and the organic titanate reacts with the substrate for up to about 10 minutes.

25. The method of claim 11, wherein the elastomeric substrate is made from an acrylonitrile butadiene or an ethylene propylene diene monomer.

26. A seal for use in an inhaler comprising:
an elastomeric substrate; and,
a titanium-containing coating on at least a portion of the substrate having a film thickness.

27. A gasket for use in an inhaler comprising:
an elastomeric substrate; and,
a titanium-containing coating on at least a portion of the substrate having a film thickness.

28. A seal for use in an inhaler made by a method comprising, in any suitable order, the acts of:
providing an elastomeric substrate in a bath comprising an alcohol and an alkaline material at a bath temperature effective for treatment;
providing ultrasonic energy at a treatment-effective frequency and power level to the bath for a time period sufficient to treat the elastomeric substrate;
rinsing the treated elastomeric substrate with de-ionized water;
drying the rinsed and treated elastomeric substrate;
providing a reactor chamber to contain the rinsed and treated elastomeric substrate;
feeding an inert gas into the reactor chamber;
applying a voltage to the reactor to create a first plasma;
feeding an organic titanate into the reactor chamber maintained at a suitable pressure;
applying a voltage to the reactor chamber generating a second plasma; and,
purging the reactor chamber.

29. A gasket for use in an inhaler made by a method comprising, in any suitable order, the acts of:
providing an elastomeric substrate in a bath comprising an alcohol and an alkaline material at a bath temperature effective for treatment;
providing ultrasonic energy at a treatment-effective frequency and power level to the bath for a time period sufficient to treat the elastomeric substrate;
rinsing the treated elastomeric substrate with de-ionized water;
drying the rinsed and treated elastomeric substrate;
providing a reactor chamber to contain the rinsed and treated elastomeric substrate;
feeding an inert gas into the reactor chamber;
applying a voltage to the reactor to create a first plasma;
feeding an organic titanate into the reactor chamber maintained at a suitable pressure;
applying a voltage to the reactor chamber generating a second plasma; and,
purging the reactor chamber.

30. A metering valve comprising:
a valve body;
a metering chamber;
a valve stem; and,
one or more stem seals comprising an elastomeric substrate and a titanium-containing coating on at least a portion of the substrate having a film thickness,
wherein the metering valve is suitable for metering a drug suspension comprising a medicament and a liquid propellant, and
wherein the medicament is a member selected from the group consisting of salmeterol, salbutamol, formoterol, ipratroprium, fluticasone, beclomethasone, budesonide, terbutaline, salts esters, solvates thereof, and combinations thereof.

31. A metered dose inhaler comprising:
a cannister in communication with a metering valve, the metering valve comprising:
a valve body;
a metering chamber;
a valve stem; and,
one or more stem seals comprising an elastomeric substrate and a titanium-containing coating on at least a portion of the substrate having a film thickness,
wherein the metering valve is suitable for metering a drug suspension comprising a medicament and a liquid propellant, and
wherein the medicament is a member selected from the group consisting of salmeterol, salbutamol, formoterol, ipratroprium, fluticasone, beclomethasone, budesonide, terbutaline, salts esters, solvates thereof, and combinations thereof.

32. A drug product comprising:
a cannister containing a drug suspension comprising a propellant and a medicament in communication with a metering valve, the metering valve comprising:
a valve body;
a metering chamber;
a valve stem; and,
one or more stem seals comprising an elastomeric substrate and a titanium-containing coating on at least a portion of the substrate having a film thickness,
wherein the metering valve is suitable for metering the drug suspension, and,
wherein the medicament is a member selected from the group consisting of salmeterol, salbutamol, formoterol, ipratroprium, fluticasone, beclomethasone, budesonide, terbutaline, salts esters, solvates thereof, and combinations thereof.
